(19) United States
(12) Patent Application Publication
(10) Pub. No.: US 2006/0188929 A1
(43) Pub. Date: Aug. 24, 2006

(54) Method capable of being automated for detection of prpres and uses thereof

(76) Inventors: Nathalie Morel, Gentilly (FR); Christophe Creminon, Orsay (FR); Jacques Grassi, Bures sur Yvette (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/520,397

(22) PCT Filed: Jul. 8, 2003
(86) PCT No.: PCT/FR03/02117

§ 371 Date: Oct. 14, 2005

(30) Foreign Application Priority Data
Jul. 9, 2002 (FR) 02/08608

(51) Int. Cl. G01N 33/53

(52) U.S. Cl. 435/7.1

(57) ABSTRACT

This detection method uses a solid support whereon plasminogen is immobilized, and includes essentially the following steps: (a) preparing the biological sample during which the latter is incubated on a homogenizing buffer which comprises an ionic or nonionic surfactant, a glucose-containing buffer, a saccharose-based buffer and a PBS buffer or in a capture buffer comprising an ionic surfactant; said step optionally comprising incubation with a proteinase K at a final concentration ranging between 1 and 8 μg/ml; (b) capturing the PrP^{res} on the solid support, which is carried out in the presence of a capture buffer, as defined above; (c) a controlled denaturation of the PrP^{res} fixed on the support comprising incubation with a denaturation buffer including at least one chaotropic agent, at a temperature ranging between room temperature and 100° C.; and (d) detecting the denatured PrP^{res} fixed on said support with a PrP protein-specific antibody.

The present invention relates to a sensitive, rapid and simple method capable of being entirely automated for detecting PrP^{res} in a biological sample and also to uses thereof.

Transmissible subacute spongiform encephalopathies (TSSEs) are caused by nonconventional transmissible agents (NCTAs), also called prions, the precise nature of which remains disputed to date. TSSEs essentially comprise Creutzfeldt-Jakob disease in humans (CJD), scrapie in sheep and goats, and bovine spongiform encephalopathy (BSE) in bovines; other encephalopathies have been demonstrated in Pelidae, in mink or in certain wild ruminants such as stags.

These diseases progress to be constantly fatal and, at the current time, no effective treatment exists.

In TSSEs, the accumulation of a host protein, PrP (or prion protein), in an abnormal form (PrP^{res}), is commonly observed, during the clinical phase of the disease, mainly in the central nervous system in the form of amorphous aggregates or of amyloid plaques. PrP^{res} copurifies with the infectiousness and its accumulation precedes the appearance of the histological lesions. In vitro, it is toxic for neurone cultures.

The two isoforms of PrP have the same amino acid sequence, but differ in their secondary structure: PrP^{res} has a significantly higher content of β-pleated sheets, whereas normal PrP (PrP^{sens}) has a greater percentage of α-helices.

The infectious isoform PrP^{res} is capable of converting the normal protein, i.e, PrP^{sens}, to an infectious protein.

Two biochemical properties generally make it possible to distinguish these two isoforms:

PrP^{res} is partially resistant to proteases, in particular to proteinase K (PK), which results in cleavage of its N-terminal end. After the action of PK, PrP^{res} is often called PrP27-30 because of the apparent molecular weight of the diglycosylated form; it is generally accepted that the PrP^{res} cleavage site is located between amino acids 89 and 90 (Prusiner et al, Cell, 1984) for the usual strains;

PrP^{res} is insoluble and aggregates in nonionic detergents, such as Triton X100 or Triton 114, forming amyloid fibers (scrapie associated fibrils, SAFs).

The normal form of the prion protein (PrP^{sens}) is, in principle, completely degraded by proteases and is entirely soluble in the presence of nonionic detergents.

To detect the presence of the infectious agent, most of the methods are based on a selective detection of the abnormal PrP (PrP^{res}), associated with the infectious agent, by taking advantage of its partial resistance to proteases and of its aggregation properties.

However, although PrP^{res} and PrP^{sens} differ in terms of their physical properties, it is in fact very difficult to develop immunoassays which make it possible to reliably differentiate the two isoforms of PrP, in particular due to the lack of PrP^{res}-specific antibodies. In fact, to date, the only antibodies available recognize either PrP^{sens} or the twvo forms of PrP (sens or res) after they have undergone a denaturation step. It is for this reasoa that, in virtually all the immunoassays dedicated to the diagnosis of TSsEs, there is a denaturation step so as to allow immunodetection of PrP^{res}.

The five major methods conventionally used for diagnosing TSSEs are:

1. histopathology, which is aimed at detecting, essentially in central nervous tissues, the lesions characteristic of TSSEs (spongiosis, vacuolization, astrogliosis, PrP amyloid plaques); it remains a reference method for confirming a clinical diagnosis. It is very specific since it makes it possible to directly observe the marks of the disease. However, it is now known that it is less sensitive than other techniques. This method has the drawback of not allowing a preclinical diagnosis, insofar as the anatomical lesions appear late in the histo)ry of the disease. In addition, it is not at all suitable for an analysis carried out in large series.

2. immunohistory chemistry, which makes it possible to detect the amyloid plaques or the deposits of PrP^{res} using PrP-specific antibodies. The sensitivity of the observation under the microscope can, in fact, be significantly increased by virtue of this approach. These techniques are certainly among the methods that are the most sensitive today, but they remain laborious and are especially used as confirmation methods.

3. detection of the amyloid fibers by electron microscopy. This method has the drawback of being relatively insensitive and laborious to implement. It has, today, been virtually abandoned.

4. bioassays, which are aimed at identifying the infectious nature of a sample. In fact, the most sensitive method for diagnosing TSSEs is, unquestionably, experimental infection in laboratory animals. This method consists in injecting an animal with a homogenate prepared from the tissue studied and in monitoring the appearance of the clinical. signs. The development of this experimental disease is confirmed using conventional techniques (histology, immunohistology, Western blotting). For obvious practical reasons, these experiments are generally carried out on rodents (mice, hamsters) but, in certain extreme cases, experimental infections have been carried out with members of the ovine race or bovines. The efficiency of the experimental transmission depends on many factors, and in particular: on the species barrier, on the amount of transmissible agent inoculated, on the strain of prion, on the sensitivity to the recipient species and on the route of inoculation. The most efficient route is the intracranial route, and then the intravenous route (10 times less efficient). The least efficient route is the oral route (100 000 times less efficient than the intracranial route). Thus, the most sensitive means for detecting the transmissible agent responsible for BSE is intracranial injection in bovines. The main drawbacks of these methods are, firstly, their laborious nature and, secondly, their duration. In fact, it takes between 300 and 700 days to carry out an experimental infection test in mice and between 3 and 10 years in bovines. The availability of transgenic mice expressing the same PrP as that of the donor species will make it possible to shorten these periods, but, in all cases, these tests will last at least three months.

5. Western blotting methods, which arem based on the immunodetection of PrP^{res} in a tissue extract, after treatment of the extract with a protease (PF, for example), so as to destroy the normal isoform of PrP (PrP^{sens}), separation of the proteins of the extract by electrophoresis, transfer onto a polymer membrane, and detection with a specific antibody that recognizes PrP (O. Schaller et al., Acta Neuropathol (Berl), 1999, 98, 437-443). For the reasons explained above, the digestion with a protease is necessary insofar as, in order to perform a Western blotting analysis, the protein is denatured, which implies that there no longer exists any difference between the normal form (PrP^{sens}) and the pathological form (PrP^{res}) of the prion protein. Digestion with PK overcomes this disadvantage since PrP^{sens} is completely digested, whereas PrP^{res} is relatively unmodified. The specificity of this approach comes, inter alia, from the fact that, under the action of proteinase K, the molecular weight of PrP^{res} is modified in a characteristic manner due to the partial degradation of the N-terminal portion of thce protein. Its sensitivity is of the same order of magnitude as that of immunohistology. The main drawback of this technique is linked to the difficulty in carrying it out, to the duration of the analysis (>8 hours) and to the fact that it is impossible to automate it.

More recently, ELISA-type assays have been described. Among these, some involve treatment of the tissue extracts with a protease; mention may be made of;

that described by Serban et al. (Neurology, 1990, 40, 110), who had developed an assay for detecting PrP^{res}. which includes immobilization of the proteins on a nitrocellulose membrane, followed by protease digestion, denaturation, and immunodetection with monoclonal antibodies;

that described by Oesch et al. (Biochemistry, 1994, 33, 5926-5931), who have proposed, in order to quantify the amount of PrP^{res}, an immunofiltracion assay for the purification of PrP^{res} (ELIFA or enzyme-linked immmunofiltration assay);

that described by Gratwohl et al., 1997, who propose an ELISA-type assay. After treatment of the samples with proteinase K and purification of PrP^{res} by centrifugation, said PrP^{res} is adsorbed onto microtitration plates and detected by means of rabbit polyclonal antibodies.

None of the abovementioned methods is truly suited to a high throughput screening and cannot be suitable for automation. After the first “mad cow crisis” in 1996 and the possible transmission of this disease to humans being taken into consideration, it was felt that there was a need to develop new diagnostic approaches that were simpler ard faster. These methods will have to make it possible to either carry out epidemiological studies on a large scale, in order to evaluate more precisely the characteristics of the epizootic, or to systematically test, in the abattoir for example, all animals before they enter into the food chain or the industrial circuits. Thus, a new generation of “fast” diagnostic tests developed, which tests are all based on the immunodetection of PrP^{res}.

In May 1998, European Commission Directorate General XXIV (consumer policy and consumer health protection) put out a worldwide invitation to tender, intended to take an inventory of the techniques capable of performing a high throughput of BSE screening and liable to rapidly give rise to an industrial development. At the end of this invitation to tender (June 1998), four tests were selected. Three of them were developed by industrial companies: Enfer Technology Ltd (Ireland) (international PCT applications WO 98/35236, in the name of Enfer Technology Ltd, and WO 93/11155, in the name cf Proteus Molecular Design Limited), Prionics (Switzerland) (international PCT application WO 99/15651) and E. G. & G. Wallac (Great Britain) (international application WO 00/29850), and the fourth was developed in two laboratories of the CEA [Atomic Energy Commission] (France). The aim of these four tests is to detect the presence of PrP^{res} in the brain of animals. They all involve treatment of the brain extracts with proteinase K in order to destroy PrP^{sens} and to allow selective measurement of PrP^{res}. The Prionics test uses the Western blotting technique in an industrialized form, whereas the tests developed by Enfer Technology and Wallac are immunoenzyme assays of the ELISA type, The test developed by the CEA involves, first, selective purification of PrP^{res}, which is then assayed by means of a sandwich assay using two monoclonal antibodies (two-site immunoenzymetric assay). This study showed that three of the tests evaluated (Prionics, Enfer and CEA) had an excellent ability to specifically detect bovines that were at the clinical stage of the disease. Moreover, the test developed by the CEA showed that it was significantly more sensitive than that of the competitors, due in particular to the PrP^{res} purification/concentration step (Moynagh (et al., Nature, 1999, 400, 105; http:/europa.eu.int/comm/dg24/health/).

Thus, the Applicant has proposed a test for quantitatively detecting PrP^{res}, which conmprises a purification step which results in a significantly more sensitive detection; this test is in particular described in international PCT application WC) 99/41280 and in a preliminary report from the European Commission Directorate General XXIV (consumer policy and consumer health protection; http://europa.eu.int/comm/dg24/health/); it has also proposed, in international PCT application WO 01/35104, a diagnostic method which, besides the possibility of purification mentioned above, uses treatment of the biological sample with a protease, so as to completely degrade PrP^{sens} under the conditions where all or some of the repeat octapeptide units of PrP^{res} are conserved; this makes it possible to detect Prpse, using a high-affinity antioctapeptide antibody. This method is very sensitive and very specific; however, the purification/concentration method comprises several steps, and in particular a centrifugation step, which prohibits any complete automation of the test.

Since 1999, these rapid tests have shown their use in the context of epidemiological studies relating to populations at risk (dead animals, destroyed as an emergency or put down because of disease). Since the beginning of 2001, they have been used on a vsry large scale, in order to test all bovines over the age of 24 or 30 months which enter into the food chain (8.5 million tests performed in 2001).

Today, the priority in the field of prion-disease diagnosis is the development of an ante-mortem and preclinical test for variant Creutzfeldt-Jakob disease. The objective is first to make blood transfusion safe and then to achieve early detection of individuals suffering from this disease in order to be able to envision setting up a treatment (which does not exist at this time) before the phase of neuroinvasion and the appearance of the first irreversible clinical signs. This necessarily implies the development of a test on a blood sample or urine sample, the only biological fluids that can readily be sampled noninvasively. This objective appears to be achievable since some publications refer to the presence of infectious prions or of PrP^{res} in the blood (Brown et al., Transfusion, 1999, 39, 1169-1178; Houston et al. The Lancet, 2000, 356, 999-1000; Schmerr et. al., J. Chromat. A., 1999, 853, 207-214) or in the urine (Shaked et al., J. Biol. Chem., 2001, 276, 31479-31482). However, the analysis of this type of sample poses analytical problems that are much more difficult to solve than those encountered in analyzing tissues known to replicate and accumulate prpres (brains, lymphoid tissues). In fact, the data available on the physiopathology of TSSEs show that, in any event, PrP^{res} is at least 100 times less concentrated in the blood or urine than in a spleen or a brain. Furthermore, it is probable that, in these media (urine, white blood cells), the biochemical properties of PrP^{res} are different from those observed in the tissues where it substantially accumulates. Its aggregation properties and its resistance to PK may in particular be very decreased. It is possible, for example, that the proteinase K treatments used to analyze a brain sample also destroy the traces of PrP^{res} contained in the blood. Consequently, in order to analyze this type of sample, strategies different from those developed to date must be developed.

One of the possible options consists in using a ligand, capable of specifically recognizing PrP^{res}. This ligand, immobilized on a suitable solid support, could make it possible to concentrate PrP^{res} in media, such as blood or urine, in which it is relatively unconcentrated. Insofar as the interaction between the ligand and PrP^{res} is really specific, protieinase K treatment will not be necessary, nor will it be necessary to make use of the aggregation properties of PrP^{res}.

This type of ligand has been described recently by the team of Adriano Aguzzi (Fischer et al., Nature, 2000, 408, 479-483; Maissen et al., The Lancet, 2001, 357, 2026-2028) and has been the subject of an international application Wo 01/23425. In that international application, in order to allow the detection of small amounts of prion, it is proposed to concentrate PrP^{res} or its PK-digestion products by treatment of the biological sample concerned with magnetic beads carrying prion-binding sites: purified plasminogen, fibrinogen, fraction I of ammonium sulfate precipitation of serum or plasma, or fraction II of ammonium sulfate precipitation of serum or plasma. The PrP^{res} is therefore first concentrated by incubation with magnetic beads carrying plasminogen or fibrinogen, and then detected by Western blotting analysis, ELISA, immunoprecipitation, BIACORE assay, immunocytochemical assay or histoblot assay after elution of the PrP^{res} from the solid support. In this method, the conditions are as follows:

sample preparation step: homogenization and centrifugation of the homogenate; it is important to use, during the first homogenization step, low concentrations of ionic detergents, followed by low-speed centrifugation (500 g for 30 minutes), whereas, in the subsequent steps, high concentrations of nonionic detergents are used; a protein concentration in the homogenate of at most 5 mg/ml is preferably obtained;

digestion with proteinase K: preferably in the presence of 50 μg/ml of PK, at 37° C., for at least half an hour;

conditions for incubation of the magnetic beads with the homogenate, in a nonionic buffer: approximately 1 and a half hours at ambient temperature;

detection conditions: in order to carry out this operation, it is first of all necessary to denature the proteins attached, which results in them detaching from the magnetic beads: the procedure is carried out in two stages: washing of the beads with a washing buffer comprising 2% Tween 20 and 2% NP-40 in PBS, and then addition of a loading buffer for the electrophoresis comprising 50 mM tris, pH 6.8, 2% SD$, 0.01% bromophenol blue and 10% glycerol, and heating at 95° C. for 5 minutes. The denatured PrP^{res} thus eluted from the solid support containing the plasminogen is then analyzed by means of Western blotting. In fact, since no antibody specific for PrP^{res}, capable of detecting it when it is bound to plasminogen, exists, it is necessary to break the plasminogen/PrP^{res} bond and therefore to denature the PrP^{res}, so as to detect it in denatured form by another method, which implies that it is detached from the beads. Such a procedure is suitable for Western blotting analysis, but not at all suitable for ELISA-type assays that use a support such as microtitration plates or magnetic beads. In fact, the conditions used in the method described in that international application Wo 01/23425 (use of SDS, in particular), due to the dissociation of the plasMinogen/PrP^{res} complex when it is denatured, means that an additional step of binding into a solid support is necessary. It should be noted, in addition, that the method described in that application does not show any ability to concentrate the PrP^{res} contained in a large volume of sample.

Because of this, the applicant gave itself the aim of providing a specific, sensitive, simple and rapid method for detecting PrP^{res}, which corresponds more thoroughly to the current objectives of the diagnosis of TSSEs than the detection methods of the prior art, in particular:

in that it is easy to use, i.e. more suitable for the conditions of routine use and therefore can be entirely automated; and

in that it is capable of purifying and concentrating PrP^{res} without making use of its PK-resistance or aggregation properties.

A subject of the present invention is a method for detecting PrP^{res} in a biological sample, using a solid support, in particular magnetic beads or micro-titration plates, on which plasminogen is immobilized, which method is characterized in that it comprises:

(a) a step which consists in preparing the biological sample, which may consist of either a tissue or cell homogenate, or of serum or plasma, or of urine, during which step this sample is incubated in a buffer selected from the group consisting of:

(i) buffers for homogenizing the biological sample comprising (1) a buffer selected froin the group consisting of buffers comprising at least one surfactant selected from the group consisting of ionic surfactants and nonionic surfactants, a glucose-containing buffer, a sucrose-based buffer and a PBS buffer and (2) optionally, a proteinase K at a final concentration of between 1 and 8 μg/ml, preferably at a final concentration of between 2 and 4 μg/ml, and

(ii) capture buffers comprising at least (1) a surfactant selected from the group consisting of ionic surfactants, and (2) optionally, a proteinase K at a final concentration of between 1 and 8 μg/ml, preferably at a final concentration of between 2 and 4 μg/ml.

Although, under the capture conditions selected in step (b) below, the plasminogen very preferentially recognizes PrP^{res}, in certain situations, prior controlled treatment with PK (i.e. during step (a) for preparing the biological sample) makes it possible to eliminate the signal associated with a residual recognition of PrP^{sens}. For this reason, it is considered that the controlled use of PK in this step is optional; moreover, in situations where it may be feared that the treatment with PK affects PrP^{res} (for example in a blood or urine sample), this PK-treatment step can be eliminated.

It should be noted that the concentration of PK used is much lower than that used in international application WO 01/23425 (50 μg/ml) or in the other tests using PK (commonly between 40 and 100 μg/ml);

(b) a step which consists in capturing PrP^{res} on said solid support, necessarily carried out in the presence of a capture buffer as defined above, without PK, i.e. in which the surfactants are exclusively ionic surfactants, by incubation of the biological sample obtained in step (a) with said support on which plasminogen is covalently immobilized; this step comprises, if necessary, prior to the incubation, a dilution of the biological sample obtained in step (a) in said capture buffer, so as to obtain the adjustment of the protein concentration, in particular when step (a) has been carried out in a homogenizing buffer.

The optimum protein concentration in the biological sample varies according to the medium studied. In the case of a brain homogenate, it is preferable for it not to iexceed approximately 2 mg/ml (corresponding to a homogenate at 2% w/v), otherwise a loss of efficiency of the capture of PrP^{res} may be observed. This limitation is probably linked to the presence in the sample of uncharacterized substances capable, themselves also, of binding to the plasminogen. It constitutes a drawback compared with other methods of concentration (for example that described in international PCT application WO 99/41280) which make it possible to treat more concentrated homogenates (homogenate at 20% w/v, i.e. approximately 20 mg/ml of protein);

(c) a step which consists of controlled denaturation of the PrP^{res} attached to said support by means of the plasminogen, comprising incubation of the PrP^{res} with a denaturing buffer comprising at least one chaotropic agent, at a temperature of between ambient temperature and 100° C.

This controlled denaturation step is compatible with maintenance of the plasminogen/PrP^{res} complex;

(d) a step which consists in detecting the denatured PrP^{res} attached to said support, with a PrP protein-specific antibody.

After capture step (b), the support. to which the PrP^{res} is possibly attached can advantageously be washed; the washing conditions, and in particular the washing buffer used, are not essential in the method according to the invention.

According to an advantageous embodiment of the method according to the invention, the ionic surfactant used in step (a) or in step (b) is selected from the group consisting of:

anionic surfactants, such as SDS (sodium dodecyl sulfate), sarkosyl (lauroylsarcosine), sodium cholate, sodium deoxycholate (DOC) or sodium tauro-cholate; and

zwitterionic surfactants such as SB 3-10 (decylsulfobetaine), SB 3-12 (dodecylsulfobetaine), SB 3-14 (tetradecylsulfobetaine), SB 3-16 (hexadecyl-sulfobetaine), CHAPS or deoxy-CHAPS.

According to another advantageous embodiment of the method according to the invention, the nonionic surfactant used in step (a) of the method according to the invention is selected from the group consisting of C12E8 (dodecyl octaethylene glycol), Triton X100, Triton X114, Tween 20, Tween 80, MEGA 9 (nonanoyl methyl glucamine), octylglucoside, LDAO (dodecyl dimethylamine oxide) or NP40.

According to another advantageous embodiment of the method according to the invention, the incubation time in step (a) is between 5 and 30 minutes at 37° C., preferably for 10 minuteslat 37° C.

According to another advantageous embodiment of the method according to the invention, the capture buffer preferably comprises sarkosyl at a final concentration of between 0.5% and 2% (w/v), even more preferably at a final concentration of sarkosyl of 1% (w/v)

According to another advantageous embodiment of the method according to the invention, the capture buffer also comprises a salt preferably selected from alkali metal salts, preferably sodium chloride, even more preferably at a concentration of between 0.15 M and 0.5 M.

According to yet another advantageous embodiment of the method according to the invention, the capture buffer also comprises a protein, and even more preferably bovine serum albumin at a concentration of 0.2 mg/ml.

According to another advantageous embodiment of the method according to the invention, the incubation time in step (b) is between 1 hour and 4 hours at ambient temperature.

According to another advantageous embodiment according to the invention, the chaotropic agent used in the controlled denaturation step (c) is selected from the group consisting of urea, a guanidine salt, such as guanidine hydrochloride or guanidine thio-cyanate, and sodium thiocyanate, or a mixture thereof.

According to another advantageous embodiment of the method according to the invention, the incubation time in step (c) is between 10 and 60 minutes, preferably either for 30 minutes at 37° C. with the microtitration plates or for 10 minutes at 100° C. with the magnetic beads.

According to another advantageous embodiment of the method according to the invention, the tracer antibody in step (d) is a polyclonal or monoclonal antibody selected from the group consisting of SAF antibodies and anti-recombinant PrP antibodies; more precisely, the SAF antibodies, and more particularly the SAF-34, SAP-53 and SAF-61 antibodies, were obtained by immunizing mice, in which the PrP gene had been knocked out, with denatured hamster SAPs (Demart et al., Biochem. Biophys. Res. Commun., 1999, 265, 652-657). The B-221, BAR 224 and BAR-233 antibodies were obtained by immunizing mice, in which the PrP gene had been knocked out, with a recombinant sheep PrP. The 8G8 antibody was obtained by immunizing mice, in which the PrP gene had been knocked out, with a recombinant human PrP (Krasemann et al., J. Immunol. Methods, 1996, 199, 109-118 and Mol. Med., 1996, 2, 725-734).

In accordance with the invention, the solid support is advantageously selected from the group consisting of magnetic heads and microtitration plates.

Surprisingly, the fact that the biological sample:

is, if necessary, homogenized in a homogenizing buffer optionally comprising PK, at very low concentrations (between 1 and 8 μg/ml),

is incubated in a capture buffer containing, as surfactant, exclusively ionic surfactants,

is brought into contact with a solid support, to which plasminogen is covalently attached,

and then is subjected to a controlled denaturation step which also surprisingly does not result in the destruction of the PrP^{res}-plasminogen bond, allows selective attachment of PrP^{res} to the solid support and direct assaying of the PrP^{res} on the solid support, without requiring additional steps.

Such a method makes it possible to perform a continuous, completely automated assay, unlike the method described in international PCT application WO 99/41280 which requires a centrifugation step. Such an assay also has a sensitivity that is at least as good as that obtained with the methods using a purification step, as described in international PCT application WO 99/41280.

A subject of the present invention is also a diagnostic kit for carrying out the method as defined above, characterized in that it comprises, in combination:

at least one homogenizing buffer as defined above,

at least one capture buffer as defined above,

at least one denaturing buffer as defined above,

a proteinase K at a final concentration of between 1 and 8 μg /ml, preferably at a final concentration of between 2 and 4 μg/ml, and

a solid support to which plasminogen is covalently attached.

Besides the above provisions, the invention comprises other provisions which will emerge from the following description, which refers to nonlimiting examples of the method according to the invention and also to the attached drawings in which:

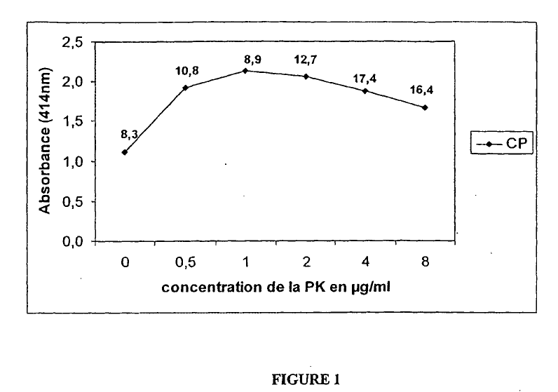

FIG. 1 illistrates the effect of the proteinase K concentration on the CP (positive control) to CN (negative control) signal ratio;

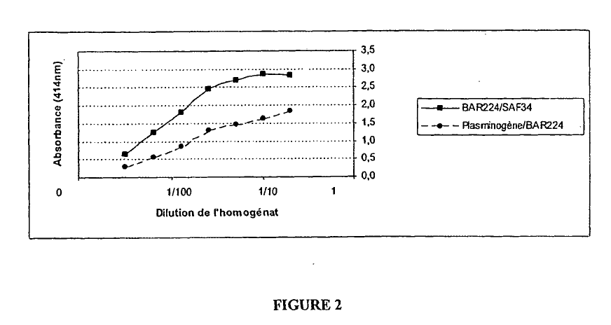

FIG. 2 represents a comparative study of the detection of sheep PrP^{res} using a conventional “sandwich” assay (BAR-224/SAF-34) or with the plasminogen/BAR224 couple;

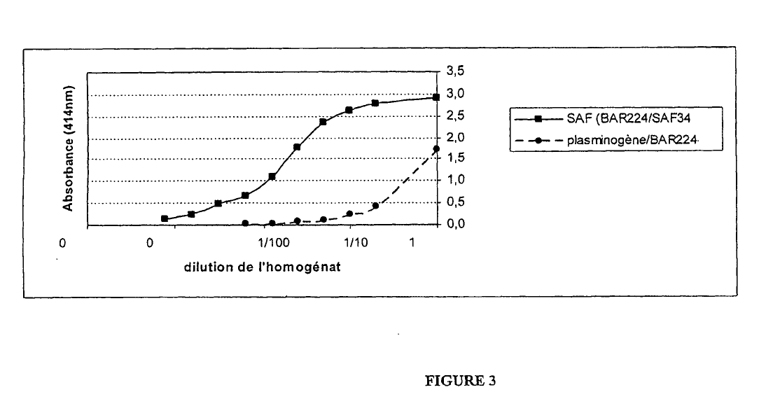

FIG. 3 represents a comparative study of the assaying of PrP^{res} from a sheep suffering from scrapie, using the technique described in international application WO 99/41280 the method according to the invention: plasminogen/BAR224 sandwich assay on a microtitration plate;

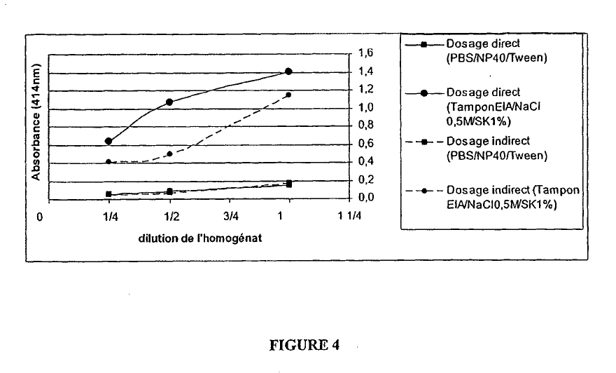

FIG. 4 represents a comparative study of the direct assaying (invention) and of the indirect assaying (method according to international application WO 01/23425) of PrP^{res} from a sheep suffering from scrapie: comparison of the conditions for capture by plasminogen immobilized or magnetic beads according to the invention or according to international application WO 01/23425;

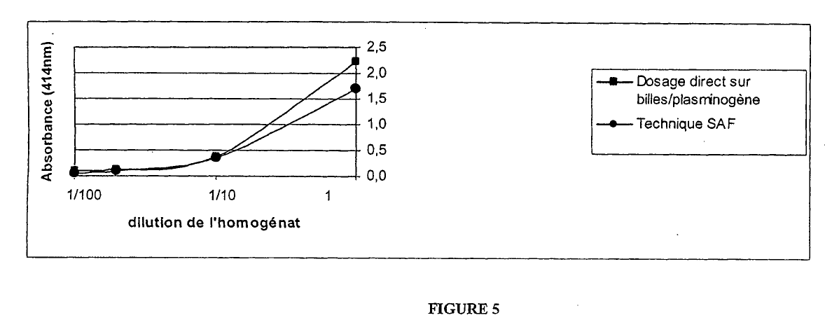

FIG. 5 illustrates the comparison of the detection of PrP^{res} using the technique consisting in preparing SAFs followed by immunometric assay (international PCT application WO 99/41280) with that using PrP^{res} capture on beads coupled to plasminogen followed by a direct assay (invention);

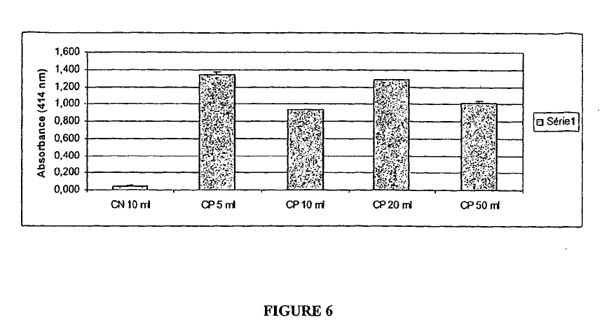

FIG. 6 represents the effect of the dilution of a homogenate of brain from a sheep suffering from scrapie on the detection of PrP^{res} by direct assaying on plasminogen coupled to magnetic beads;

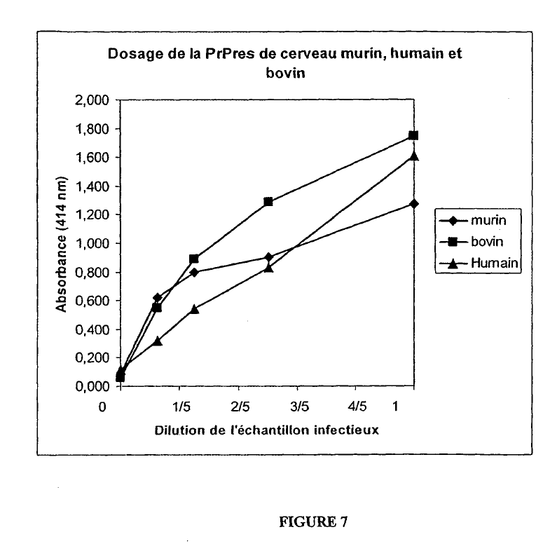

FIG. 7 represegts dilution curves for brain from mice, cows and humans suffering from a TSSE. It illustrates the ability of the method according to the invention to provide a diagnosis for all the TSSEs.

### EXAMPLE 1

Method of Detection According to the Invention: Optimization of Various Parameters

1. Coupling of Plasminogen to a Covalink NH Solid Support:

The plasminogen is immobilized covalently at the surface of Covalink NH microtitration plates (Nunc) using a homobifunctional coupling agent, disuccinimidyl suberate (DSS). 100 μl of a DSS solution (12.5 mg of DSS dissolved in 50 ml of DMSO and 50 ml of 50 mM carbonate buffer pH 9.5) are incubated at the surface of the Covalink NH wells for 1 hour at ambient temperature.

The wells are washed 3 times with distilled water, and then 100 μl of a 2.5 μg/ml plasminogen solution in 50 mM carbonate buffer, pH 9.5, are incubated at the surface of the wells overnight at ambient temperature. The wells are emptied and saturated with EIA buffer. (0.1 M phosphate buffer, pH 7.4, containing 0.15 M NaCl, 0.1% BSA and 0.01% sodium azide).

2. Preparation of the Sample (step (a) of the Method) and Capture of PrP^{res} on the Covalink NH Microtitration Plates Containing the Plasminogen (step (b) of the Method)

A. Conditions for preparing the sample with a view to capture

25 μl of a homogenate of brain from a sheep suffering from scrapie (CP=positive control) or a normal sheep (CN=negative control) are incubated with 225 μl of EIA buffer, pH 7,4, comprising an ionic surfactant and proteinase K at a final concentration of 1 μg/ml, for 10 minutes at 37° C., and then 10 μl of 100 mM Pefabloc™ (protease inhibitor corresponding to [4-(2-aminoethyl)benzenesulfonyl] fluoride HCl) are added. 100 μl of sample are deposited in the wells of the Covalink NH microtitration plate containing the plasminogen, and are incubated for 2 hours at ambient temperature.

B. Effect of surtactants on the capture of PrP^{res} by the plasminogen immobilized on a Covalink NH solid support

Table I below illustrates the results obtained using the BAR-224 antibody to detect the PrP^{res} associated with the plasminogen after controlled denaturation by treatment with guanidine/HCl.

This table I describes the conditions tested on the capture of PrP^{res} by the plasminogen; this table gives details of the effect of various surfactants: Sarkosyl=SK, Triton X100=T, NP40=Nonidet P40, Tween 20=Tween and sodium dodecyl sulfate=SDS.

TABLE I
Effect of detergents on the capture of PrP^{res} of brain from
a sheep suffering from scrapie by plasminogen immobilized
on a Covalink NH microtitration plate solid support

| Incubation buffer composition | | CN | CP | CP/CN |
|---|---|---|---|---|
| EIA buffer + 0.5% SK | | 0.066 | 2.435 | 37.17 |
| EIA buffer + 0.5% SK + 0.5% T | | 0.054 | 1.857 | 34.38 |
| EIA buffer + 0.5% SK + 1% T | | 0.006 | 0.837 | 139.42 |
| EIA buffer + 0.5% SK + 2% T | | 0.000 | 0.550 | — |
| EIA buffer + 1% SK | | 0.035 | 1.671 | 48.42 |
| EIA buffer + 1% SK + 0.5% T | | 0.079 | 1.942 | 24.58 |
| EIA buffer + 1% SK + 1% T | | 0.097 | 1.997 | 20.58 |
| EIA buffer + 1% SK + 2% T | | 0.003 | 0.851 | 340.20 |
| EIA buffer + 1.5% SK | | 0.020 | 1.428 | 71.40 |
| EIA buffer + 1.5% SK + 0.5% T | | 0.103 | 1.725 | 16.82 |
| EIA buffer + 1.5% SK + 1% T | | 0.062 | 1.976 | 32.13 |
| EIA buffer + 2% SK | | 0.003 | 0.804 | 267.83 |
| EIA buffer + 2% SK + 0.5% T | | 0.022 | 1.430 | 66.49 |
| EIA buffer + 2% SK + 1% T | | 0.026 | 1.731 | 67.88 |
| EIA buffer + 2% SK + 2% T | * | 0.037 | 1.88 | 50.81 |
| EIA buffer + 1% T | | 0.000 | 0.027 | — |
| EIA buffer + 2% T | | 0.000 | 0.354 | — |
| EIA buffer + 4% T | | 0.006 | 0.936 | 170.18 |
| EIA buffer + 10% T | * | 0 | 1.018 | — |
| EIA buffer + 15% T | * | 0 | 0.305 | — |
| EIA buffer + 0.5% SDS | | 0.017 | 0.095 | 5.59 |
| EIA buffer + 1% SDS | | 0.000 | 0.008 | — |
| EIA buffer + 3% NP40 | | 0.001 | 0.779 | 779.00 |
| EIA buffer + 3% NP40 + 3% Tween | | 0.000 | 0.905 | — |
| EIA buffer + 6% NP40 | | 0.013 | 0.801 | 64.08 |
| PBS + 3% NP40 + 3% Tween | [symbol] | 0.004 | 0.526 | 150.29 |
| EIA buffer + 1% DOC | * | 0.03 | 0.014 | 0.47 |
| EIA buffer + 1% SK + DOC | | 0.011 | 0.974 | 88.55 |

*: Results obtained in a different experiment and standardized relative to the result obtained with the EIA buffer + 1% SK [symbol]
: Conditions of international application WO 01/23425 used for the capture of PrP^{res} by plasminogen.
CN: Negative control
CP: Positive control
EIA buffer: 0.1 M phosphate buffer, pH 7.4 + 0.15 M
NaCl + 0.1% BSA + 0.01% sodium azide.

The capture conditions selected for the remainder of the assays are: EIA buffer+1% SK, even though, in table I above, other conditions give a slightly higher CP/CN ratio, because these conditions provide a high CP-CN differential and because, in other experiments, the results were reversed, the CN values varying.

In the absence of surfactant, no specific capture is observed (binding of PrP^{sens} is even observed).

The best results re obtained using sarkosyl as surfactant.

C. Effects of PH and of NaCl concentration on the capture of PrP^{res} by plasminogen immobilized on a Covalink NH solid support

25 μl of a homocenate of brain fr(om a sheep suffering from scrapie or a normal sheep ark incubated with 225 μl of EIA buier containing various NaCl concentrations and at various values and comprising sarkosyl at a final concentration (w/v) of 1% and proteinase K at a final concentration of 1 μg/ml, for 10 minutes at 37° C., and then 10 μl of 100 mm Pefabloc™ are added. The procedure is then carried out as described previously,

Table II gives the results obtained.

TABLE II
Effect of pH and of NaCl concentration on the capture of
PrP^{res} of brain from a sheep suffering from scrapie by
plasminogen immobilized on a Covalink NH microtitration plate

| Incubation buffer composition | | CN | CP | CP/CN |
|---|---|---|---|---|
| EIA buffer pH 6 + 0.15 M NaCl + 1% SK | | 0.032 | 0.515 | 16.33 |
| EIA buffer pH 6 + 0.3 M NaCl + 1% SK | * | 0.033 | 0.559 | 16.94 |
| EIA buffer pH 6 + 0.5 M NaCl + 1% SK | * | 0.058 | 0.499 | 8.60 |
| EIA buffer pH 6 + 0.8 M NaCl + 1% SK | * | 0.168 | 0.727 | 4.33 |
| EIA buffer pH 6.5 + 0.15 M NaCl + 1% SK | | 0.080 | 0.541 | 6.76 |
| EIA buffer pH 7 + 0.15 M NaCl + 1% SK | | 0.090 | 0.561 | 6.23 |
| EIA buffer pH 7.4 + 1% SK | | 0.093 | 0.454 | 4.91 |
| EIA buffer pH 8 + 0.15 M NaCl + 1% SK | | 0.086 | 0.326 | 3.78 |
| EIA buffer pH 7.4 + 1% SK | | 0.068 | 0.076 | 1.12 |
| EIA buffer pH 7.4 + 0.5 M NaCl + 1% SK | | 0.051 | 0.722 | 14.15 |
| EIA buffer pH 7.4 + 1 M NaCl + 1% SK | | 0.100 | 0.518 | 5.20 |

*: Results obtained in a different experiment and standardized relative to the result obtained with the EIA buffer + 1% SK
EIA buffer: 0.1 M phosphate buffer, pH 7.4 + 0.1% BSA + 0.01% sodium azide

The PrP^{res} capture conditions preferably selected are as follows: EIA buffer+0.5 M NaCl+1% SK.

D. Effect of proteinase K concentration on the CP/CN ratio

25 μl of a homogenate of brain from a sheep suffering from scrapie or a normal sheep are incubated with 225 μl of EIA buffer, pH 7.4, comprising 0.5 M NaCl, a final concentration of 1% of sarkosyl and proteinase K at various concentrations, 0, 0.5, 1, 2, 4 and 8 μg/ml final concentration, for 10 minutes at 37° C., and then 10 μl of 100 ImM Pefabloc™ are added. The procedure is then carried out as previously described.

Table III and FIG. 1 give the results obtained.

TABLE III
Optimization of the proteinase K (PK) concentration

| PK concentration in μg/ml | CN | CP | CP/CN |
|---|---|---|---|
| 0 | 0.133 | 1.107 | 8.32 |
| 0.5 | 0.178 | 1.921 | 10.79 |
| 1 | 0.240 | 2.125 | 8.87 |
| 2 | 0.162 | 2.064 | 12.74 |
| 4 | 0.108 | 1.871 | 17.40 |
| 8 | 0.103 | 1.661 | 16.20 |

These results show that PK at low concentration (2 to 4 μg/ml) improves the CP/CN ratio, while at the same time conserving a good CP signal.

3. Controlled Denaturation of PrP^{res}, Under Conditions Where the Plasminogen/PrP^{res} Complex is not Dissociated

A. Preferred denaturation conditions

After reaction for 2 hours at ambient temperature, the wells are washed and then incubated with 100 μl of guanidine/HCl, 8 M, fot 30 min at 37° C.

B. Effect of the denaturing agent used after capture and before detection of PrP^{res}, with a labeled antibody

25 μl of a homogenate of brain from a sheep suffering from scrapie and a normal sheep are incubated with 225 μl of EIA buffer, pH 7.4, comprising 0.5 M NaCl, 1% of sarkosyl and proteinase K at a final concentration of 1 μg/ml, for 10 minutes at 37° C., and then 10 μl of 100 mM Pefabloc™ are added. 100 μl are deposited in the wells of a microtitration plate containing immobilized plasminogen.

After reaction for 2 hours at ambient temperature, the wells are, washed and then incubated with 100 μl of various denaturing agents for 30 min at 37° C.

The wells are again washed and then incubated with a tracer antibody, BAR224 at 5 Ellman units/ml, for 2 hours at ambient temperature.

After washing, 200 μl of a visualizing solution (Ellman's reagent) are added. The absorbance at 414 nm of the wells is meausred after reaction for 30 min.

Table IV gives the results obtained.

TABLE IV
Effect of the denaturing agent used after capture and before
detection of sheep PrP^{res} with a labeled antibody

| Denaturing agents | | CN | CP | CP/CN |
|---|---|---|---|---|
| Urea 2 M | | 0.056 | 0.082 | 1.46 |
| Urea 4 M | | 0.055 | 0.116 | 2.11 |
| Urea 8 M | | 0.056 | 0.295 | 5.27 |
| Guanidine/HCl 2 M | | 0.108 | 0.12 | 1.11 |
| Guanidine/HCl 4 M | | 0.083 | 0.2 | 2.41 |
| Guanidine/HCl 8 M | | 0.122 | 1.445 | 11.84 |
| NaSCN 2 M | | 0.118 | 0.09 | 0.76 |
| NaSCN 4 M | | 0.054 | 0.103 | 1.91 |
| NaSCN 8 M | | 0.025 | 0.918 | 36.72 |
| Guanidine/SCN 2 M | * | 0.072 | 0.347 | 4.82 |
| Guanidine/SCN 4 M | * | 0.024 | 0.394 | 16.42 |
| Guanidine/SCN 6 M | * | 0.016 | 0.881 | 55.06 |
| NaNO₃ 2 M | | 0.103 | 0.097 | 0.94 |
| NaNO₃ 4 M | | 0.084 | 0.09 | 1.07 |
| NaOH 1 M | | 0.038 | 0.01 | 0.26 |
| NaOH 0.5 M | | 0.023 | 0.074 | 3.22 |
| NaOH 0.1 M | | 0.031 | 0.175 | 5.65 |
| HCl 1 M | | 0.092 | 0.125 | 1.36 |
| HCl 0.5 M | | 0.079 | 0.136 | 1.72 |
| HCl 0.1 M | | 0.057 | 0.071 | 1.25 |
| NaCl 2 M | | 0.085 | 0.07 | 0.82 |
| NaCl 4 M | | 0.063 | 0.064 | 1.02 |
| 50% HFIP | | 0.026 | 0.309 | 11.88 |
| Methanol | | 0.021 | 0.027 | 1.29 |
| Isopropanol | | 0.059 | 0.049 | 0.83 |
| Ethanol | | 0.042 | 0.035 | 0.83 |
| 50% Acetonitrile | | 0.02 | 0.023 | 1.15 |
| 50% DMSO | | 0.014 | 0.02 | 1.43 |
| 0.5% SDS | | 0.003 | −0.006 | — |
| 1% SDS | | 0.001 | 0 | 0.00 |
| 5% SDS | | −0.004 | −0.007 | 1.75 |
| 10% SDS | | −0.008 | −0.009 | 1.13 |
| 5% DOC | | 0.088 | 0.09 | 1.02 |
| 10% DOC | | 0.107 | 0.094 | 0.88 |
| 20% TX-100 | | 0.021 | 0.021 | 1.00 |
| 5% SK | | −0.011 | −0.012 | 1.09 |
| 10% SK | | 0.011 | −0.013 | — |
| 20% SK | | 0.019 | 0.005 | 0.26 |
| 2% CHAPS | | 0.014 | 0.012 | 0.86 |
| 0.1 M citrate buffer, pH 3.5 | | 0.017 | 0.022 | 1.29 |
| 0.1 M glycine buffer, pH 3.5 | | 0.013 | −0.001 | — |
| 10% HFIP | | −0.004 | −0.002 | 0.50 |
| EIA buffer | | 0.016 | −0.005 | — |

*: Results obtained with a different experiment and standardized relative to the result obtained with the EIA buffer + 1% SK

Only certain conditions tested give a good CP signal: it is always stronq chaotropic agents which give good detection of PrP^{res}, such as urea, guanidine hydrochloride, guanidine, thiocyanate and sodium thiocyanate.

4. Detection of PrP^{res} Complexed with Plasminogen Directly on the Solid Support: Selection of the Visualizing Antibody

The procedure is carried out as described in point 3., using 8 M guanidine/HCl as denaturing agent.

Eight antibodies were tested at a concentration of 5 Ellman units/ml: SAF34, SAF53, SAF61, 8G8, BAR221, BAR224, BAR231 and BAR233.

Table V gives the results obtained.

TABLE V
Selection of the tracer antibody for detecting sheep PrP^{res}

| Tracer | CN | CP | CP/CN |
|---|---|---|---|
| SAF34 | 0.082 | 2.163 | 26.37 |
| SAF53 | 0.381 | 2.285 | 6.01 |
| SAF61 | 0.372 | 2.385 | 6.41 |
| 8G8 | 0.160 | 1.014 | 6.36 |
| BAR221 | 0.039 | 1.133 | 29.04 |
| BAR224 | 0.015 | 2.121 | 146.24 |
| BAR231 | 0.020 | 0.153 | 7.85 |
| BAR233 | 0.042 | 0.767 | 25.98 |

The tracer antibody BAR224 gites the best CP/CN ratio and also a good CP signal for detecting sheep PrP^{res}.

### EXAMPLE 2

Comparative Study of the Detection of Sheep PrP^{res} Using a Conventional “Sandwich” Assay (BAR224/SAF34) or with the Plasminogen/BAR224 Couple

This study made it possible to compare the detection sensitivity of the two types of sandwich. A preparation of PrP^{res} (SAF) was obtained from a brain of a sheep suffering from scrappie:

For the BAR22/SAF6b 34 sandwich assay: the SAF preparations (according to the rapid SAF protocol as described in international PCT application WO 99/41280) from 250 μl of homogenate of brain from a sheep suffering from scrapie or a normal sheep are taken up and denatured with 25 μl or a denaturing buffer (buffer C, as defined in international PCT application WO 99/41280) for 10 min at100° C. The pellets are taken up with 250 μl of EIA buffer and diluted successively in EIA buffer. The dilutions are deposited in the wells of a microtitration plate containing the BAR224 antibody. After reaction for 2 hours at ambient temperature, the wells are washed and then incubated with 100 μl of the SAF34 tracer antibody (at 5 EU/ml) for 2 hours at ambient temperature. After washing, 200 μl of the visualizing solution are added. The absorbance at 414 nm is measured after reaction for 30 minutes.

For the plasminogen/BAR224 sandwich assay: the SAF preparations (identical to above) are taken up with 250 μl of EIA buffer containing a final concentration of 4 mM Pefabloc™, and then treated by ultrasound until the pellet has dissolved. Successive dilutions are then carried in EIA buffer comprising Pefabloc™. The dilutions are deposited onto a solid support (microtitration plate) containing plasminogen. After incubation for 2 hours at ambient temperature, the wells are washed; after the washing step, the PrP^{res} is denatured in a controlled manner with guanidine/HCl (8 M, 30 minutes at 37° C. and the various wells are then incubated with 100 μl of the BAR224 tracer antibody (at 5 EU/ml) for 2 hours at ambient temperature. After washing, 200 μl of visualizing solution are added. The absorbance at 414 nm is measured after reaction for 30 minutes.

FIG. 2 gives the results obtained and shows that the two systems exhibit a comparable sensitivity with a slight advantage for the entirely immunological assay.

### EXAMPLE 3

Comparative Study of the Assaying of PrP^{res} from a Sheep Suffering from Scrapie Using the Technique According to International PCT Application WO 99/41280 and the Plasminogen/BAR224 Sandwich Assay on a Micro-Titration Plate, According to the Invention

In this second experiment, the sensitivity of the two methods are compared while including, for the method according to application WO 91/41280, the SAF preparation technique and for the plasminogen technique, the dilution than must be carried out before capture, in particular so as to obtain a protein concentration in the homogenate of 20 mq/ml (2% w/v).

A homogenate containing 20% of brain from a sheep suffering from scrapie is diluted in a normal sheep brain homogenate (1/5, 1/10, 1/20, 1/40, 1/80, 1/160 and 1/320 dilution) not diluted.

In the case of the test according to international application WO 99/41220, the SAFs are prepared from 250 μl of homogenate. For the detection part, the BAR224 and SAF34, antibodies are used as capture antibody and tracer antibody, respectively.

In the case of the test according to the invention:

the plasminogen is immobilized on the micro-titration plates as specified in example 1;

the assay is carried out with 25 μl of homogenate, using, as capture buffer, EIA buffer comprising 0.5 M NaCl, 1% sarkosyl and 2.5 μg/ml of proteinase K, 8M guanidine/HCl as denaturing agent, and BAR224 as tracer antibody.

The results are given in FIG. 3 and show that the test according to international PCT application WO 99/41280 has a very clear advantage in terms of sensitivity because it processes 250 μl of 20% homogenate, i.e. 50 mg of brain tissue, instead of 25 μl of the same 20% homogenate, 5 mg, for the test according to the invention. This disadvantage is the result of the use of microtitration plates, because the volume of 2% homogenate processed is limited (a maximum of 300 μl). However, this disadvantage disappears when magnetic beads, which make it possible to process very large volumes (at least 50 ml) are used.

### EXAMPLE 4

Comparative Study of the Direct Assay According to the Invention with an Indirect Assay of PrP^{res} from a Sheep Suffering from Scrapie, Attached to Plasminogen Immobilized on Magnetic Beads. Comparison with the Capture conditions used in International Application WO 99/41280

100 μg of plasminogen were coupled to 1 ml of magnetic beads (Dynal M-280) according to the method described by the manufacturer.

25 μl of a homogenate containing 20% of brain from a sheep suffering from scrapie or a normal sheep are incubated:

(1) either with 225 μl of capture buffer, as described above,

(2) or with 225 μl of PBS comprising 3% of NP-40 and 3% of Tween-20 (conditions described in international PCT application WO 01/23412).

10 μl of beads, coupled to plasminogen, are added to each sample and then incubated for 2 hours at ambient temperature with rotation. The beads are washed 3 times:

either (1) with EIA buffer comprising 1% of Tween 20,

or (2) with PBS comprising 2% of NP.40 and 2% of Tween 20.

After a wash with PBS, 30 μl of 6M guanidine/HCl are added for the direct assay and 30 μl of 6M urea and 0.25% sarkosyl are added for the indirect assay, then incubation is carried out for 10 minutes at 100° C.

The results are given in FIG. 4:

In the case of the direct assay, after the denaturation step, the plasminogen-coupled beads are washed and then incubated with 500 μl of BAR224 tracer in EIA buffer/1% Tween 20 (at 5 EU/ml) for 2 hours at ambient temperature with agitation. The beads are then washed twice with EIA buffer/1% Tween 20 and once with PBS, before adding 600 μl of Ellman's reagent. After reaction for 30 minutes, 200 μl of reaction miedium are removed and the absorbance at 412 nm is measured.

In the case of the indirect assay, after the denaturation step, the denatured PrP eluted from the plasminogen is taken up with 300 μl of EIA buffer and measured using a BAR224/SAF34 “sandwich” assay.

It emerges from the example that the capture conditions according to the present invention make it possible to obtain results that are significantly superior to those obtained with the capture conditions of international application WO 01/23125. It will also be noted that the directs assay, which is simpler, is also more sensitive.

### EXAMPLE 5 :

Comparison ot the Detection of PrP^{res} using the SAF Preparation Technique Folloed by an Immuno-Metric Assay (Method According to International Application WO 99/41280) with that using the Capture of PrP^{res} on Plasminogen-Coupled Beads Followed by a Direct Assay

In the case of the PrP^{res} assay according to the method described in internation PCT application WO 99/41280, the SAFs are prepared from 500 μl of homogenate containing 20% of brain from a normal sheep or a sheep suffering from scrapie (diluted 1/10, 1/50 and 1/100 in a normal sheep brain homogenate, or not diluted). The SAF pellets are taken up and denatured with 50 μl of denaturing buffer (buffer C, as defined in international PCT application WO 99/41280) for 10 minutes at 100° C. The amount of PrP is then measured with the BIO-RAD Platelia™ BSE detection kit (ref. 51103) (immunoenzymne kit in vitro detection of PrP^{res} after purification according to the method described in international PCT application WO 99/41280).

In the case of the assay using plasminogen coupled to magnetic beads, the 500 μl of homogenate (the same as above) are diluted 10 times in EIA buffer comprising 0.5 M NaCl and 1% of sarkosyl, and then incubated with 30 μl of beads containing the immobilized plasminogen for 3 hours at ambient temperature. After washing, a controlled denaturation is carried out by treatment with a guanidine/HCl solution for 10 minutes at 100° C. After 3 washes with EIA buffer/1% Tween 20 ane one wash with PBS, the beads are incubated with the BAR224 tracer in EIA buffer for 2 hours at ambient temperature. The beads are again washed 3 times with EIA buffer/1% +ween 20 and once with PBS before adding the visalizing solution (Ellman's reagent).

The results are in FIG. 5, which shows that the plasminogen technique appears to be at least as sensitive as the test according to international PCT application Wo 99/41280. The use of magnetic beads makes it possible to work with a larger volume and to compensate for the disadvantage associated with the need to dilute the homogenate before capture with the plasminogen.

### EXAMPLE 6

Effect of the Dilution of a Homogenate of Bbrain from a Sheep Suffering from Scrapie on the Detection of PrP^{res} by Direct Assay on Plasminogen Coupled to Magnetic Beads. Demonstration of the Ability of the Method to Concentrate PrP^{res} Diluted in a Large Volume of Sample

500 μl of a homogenate of brain from a sheep suffering from scrapie are diluted in 5, 10, 20 and 50 ml of EIA buffer, pH 7 comprisinlg 0.5 M NaCl and 1% of sarkosyl, and then incubated with 30 μl of plasminogen-coupled beads for 4 hours at ambient temperature. The procedure is then carried out as described above.

The results are given in FIG. 6, which shows the ability of the method according to the invention to concentrate dilute PrP^{res}.

### EXAMPLE 7

Application of the Invention to the Detection of PrP^{res} in Homogenates of Brains fromm Mice, Cows and Humans Suffering from a TSSE

Homogenates at 20% (w/v) obtained from a brain of a mouse (infected with a sheep scrapie strain) of a brain from a bovine (infected with BSE) or from a human brain (infected with Creutzfeldt-Jakob disease) were diluted to a concentration of 1% (w/v) in the capture buffer (ETA buffer comprising 0.5 M NaCl and 1% (v/v) sarkosyl, final concentration). These homogenates were then brought into contact with magnetic beads containing immobilized plasminogen, and analyzed under the conditions described in example 4.

More precisely, 850 μl of EIA buffer/0.5 M NaCl+50 μl of 10% SK+10% of plasminogen-coupled magnetic beads are added to 50 μl of a negative or positive control brain homogenate (diluted in a negative homogenate+50 μl of 10% SK, or not diluted); incubation is carried out for 2 hours 30 min at ambient temperature with rotation, the beads are then washed 3 times with EIA bufker comprising 1% of Tween 20, and then once with PBS. The controlled denaturation is carried out in the. presence of 50 μl of 4M guanidine thiocyanate (Gn/SCN) at 100° C. for 8 min. After the denaturation step, the beads are washed in PBS and then incubated with 500 μl of tracer antibody for 2 h at ambient temperature, with agitation (at 5 EU/ml). The beads are then washed twice with ETA buffer/1% Tween 20 and once with PBS, before adding 1 ml of Ellman's reagent. After reaction for 20 min, 200 μl of reaction medium are removed and the absorbance at 412 nm is measured.

This experiment (FIG. 7) shows that the test described functions, with species other than sheep and can detect prion strains other than scrapie strains.

1. A method for detecting PrP^{res} in a biological sample, using a solid support, in particular microtitration plates or magnetic beads, on which plasminogen is immobilized, which method comprises:
(a) a step of preparing the biological sample. during which step this sample is incubated in a buffer selected from the group consisting of:
(i) buffers for homogenizing the biological sample comprising (1) a buffer selected from the group consisting of buffers comprising at least one surfactant selected from the group consisting of ionic surfactants and nonionic surfactants, a glucose-containing buffer, a sucrose-based buffer and a PBS buffer and (2) optionally, a proteinase K at a final concentration of between 1 and 8 μg/ml, and
(ii) capture buffers comprising at least (1) a surfactant selected from the group consisting of ionic surfactants, and (2) optionally, a proteinase K at a final concentration of between 1 and 8 μg/ml,
(b) a step of capturing PrP^{res} on said solid support, carried out in the presence of a capture buffer as defined above, without PK, by incubation of the biological sample obtained in step (a) with said support on which plasminogen is covalently immobilized;
(c) a step of controlled denaturation of the PrP^{res} attached to said support by means of the plasminogen, comprising incubation of the PrP^{res} with a denaturing buffer comprising at least one chaotropic agent, at a temperature of between ambient temperature and 100° C., and
(d) a step of detecting the denatured PrP^{res} attached to said support, with a PrP protein-specific antibody.

2. The method as claimed in claim 1, wherein the ionic surfactant used in step (a) or in step (b) is selected from the group consisting of:
anionic surfactants, such as SDS (sodium dodecyl sulfate), sarkosyl (lauroylsarcosine), sodium cholate, sodium deoxycholate (DOC) or sodium taurocholate; and
zwitterionic surfactants such as SB 3-10 (decylsulfobetaine), SB 3-12 (dodecylsulfobetaine), SB 3-14 (tetradecylsulfobetaine), SB 3-16 (hexadecyl-sulfobetaine), CHAPS or deoxy-CHAPS.

3. The method as claimed in claim 1, wherein the nonionic surfactant used in step (a) is selected from the group consisting of C12E8 (dodecyl octaethylene glycol), Triton X100, Triton X114, Tween 20, Tween 80, MEGA 9 (nonanoyl methyl glucamine), octylglucoside, LDAO (dodecyl dimethylamine oxide) or NP40.

4. The method as claimed in claim 1, wherein the incubation time in step (a) is between 5 and 30 minutes at 37° C.

5. The method as claimed in claim 1, wherein the capture buffer comprises sarkosyl at a final concentration of between 0.5% and 2% (w/v).

6. The method as claimed in claim 1, wherein the capture buffer also comprises a salt selected from alkali metal salts.

7. The method as claimed in claim 6, wherein said salt is sodium chloride, at a concentration of between 0.15 M and 0.5 M.

8. The method as claimed in claim 1, wherein the capture buffer also comprises a protein.

9. The method as claimed in claim 1. wherein the incubation time in step (b) is between 1 hour and 4 hours at ambient temperature.

10. The method as claimed in claim 1, wherein step (b) also comprises, if necessary, prior to said incubation, a dilution of the biological sample obtained in step (a) in said capture buffer, so as to obtain the adjustment of the protein concentration.

11. The method as claimed in claim 1, wherein the chaotropic agent used in the controlled denaturation step (c) is selected from the group consisting of urea, a guanidine salt, such as guanidine hydrochloride or guanidine thiocyanate, and sodium thiocyanate, or a mixture thereof.

12. The method as claimed in claim 1, wherein the incubation time in step (c) is between 10 and 60 minutes.

13. The method as claimed in claim 1. wherein the tracer antibody in step (d) is selected from the group consisting of SAF antibodies and anti-recombinant PrP antibodies.

14. A diagnostic kit for detecting PrP^{res} in a biological sample comprising, in combination:
at least one buffer for homogenizing the biological sample comprising (1) a buffer selected from the group consisting of buffers comprising at least one surfactant selected from the grou consisting of ionic surfactants and nonionic surfactants, a glucose-containing buffer, a sucrose-based buffer and a PBS buffer and (2) optionally, a proteinase K at a final concentration of between 1 and 8 μg/ml,
at least one capture buffer comprising at least (1) a surfactant selected from the group consisting of ionic surfactants, and (2) optionally. a proteinase K at a final concentration of between 1 and 8 μg/ml,
at least one denaturing buffer comprising at least one chaotropic agent,
a proteinase K at a final concentration of between 1 and 8 μg/ml, and
a solid support to which plasminogen is covalently attached.

15. The method as claimed in claim 1, wherein the proteinase K in the homogenizing buffer is at a final concentration of between 2 and 4 μg/ml.

16. The method as claimed in claim 5, wherein the final concentration of sarkosyl is 1% (w/v).

17. The method as claimed in claim 8, wherein the protein in the capture buffer includes bovine serum albumin at a concentration of 0.2 mg/ml.

18. The method as claimed in claim 12, wherein the incubation time is either for 30 minutes at 37° C. with the microtitration plates or for 10 minutes at 100° C. with the magnetic beads.

19. The diagnostic kit as claimed in claim 14, wherein the proteinase K in the homogenizing buffer is at a final concentration of between 2 and 4 μg/ml.

20. The diagnostic kit as claimed in claim 14, wherein the proteinase K in the capture buffer is at a final concentration of between 2 and 4 μg/ml.
